(19) United States
(12) Patent Application Publication
(10) Pub. No.: US 2005/0074484 A1
(43) Pub. Date: Apr. 7, 2005

(54) Product for administration of active agents to different areas of the skin

(76) Inventors: Roderico B. Estanislao, Laguna (PH); Danilo L. Lambino, Kogarah (AU); Connie Lim, Woodland Hills, CA (US); Maki Sugimoto, Sukagawa (JP)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/681,053

(22) Filed: Oct. 7, 2003

(51) Int. Cl. A61K 9/00; A61K 9/70

(52) U.S. Cl. 424/443

(57) ABSTRACT

The present invention relates to a product including a first water insoluble substrate and a second water insoluble substrate, wherein (a) the first water insoluble substrate and the second water insoluble substrate are of different shape; (b) the first water insoluble substrate comprises a first active agent; and (c) the second water insoluble substrate includes a second active agent but does not include the first active agent.

### FIELD OF THE INVENTION

The present invention relates to a product and the uses thereof to treat the human face.

### BACKGROUND OF THE INVENTION

Facial mask products have been used for many years to cosmetically treat the skin of the human face. Consumers typically use facial mask products for treatment of various skin conditions as well as to improve the physical appearance and texture of the facial skin. Traditionally, facial mask products have been homogenous compositions that deliver the same benefit to all regions of the face. Different regions of the face, however, may have different cosmetic needs (e.g., the forehead region of the face has differing needs from the mouth region of the face).

Therefore, there is a need for a facial mask product that is capable of addressing the differing needs of the multiple facial regions.

### SUMMARY OF THE INVENTION

In one aspect, the present invention relates to a product including a first water insoluble substrate and a second water insoluble substrate, wherein (a) the first water insoluble substrate and the second water insoluble substrate are of different shape; (b) the first water insoluble substrate includes a first active agent; and (c) the second water insoluble substrate includes a second active agent but does not include the first active agent.

In another aspect, the present invention features a method of applying a first active agent and a second active agent to the skin where the method includes the steps of applying a first water-insoluble substrate including the first active agent and a second water-insoluble substrate including a second active agent to the skin, wherein the second water-insoluble substrate does not include the first active agent.

### BRIEF DESCRIPTION OF THE FIGURES

FIG. 1 is a plane view of a “T” shaped water-insoluble substrate of the present invention for application to the forehead.

FIG. 2 is a plane view of a “U” shaped water-insoluble substrate of the present invention for application proximate to the mouth area.

FIG. 3 depicts the application of both the “T” shaped water-insoluble substrate of the present invention and the “U” shaped water-insoluble substrate of the present invention to the face.

### DETAILED DESCRIPTION OF THE INVENTION

It is believed that one of ordinary skill in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Whenever used, any percentage is weight by weight (% w/w) unless otherwise indicated.

Product

The product of the present invention contains two or more water-insoluble substrates. In one embodiment, the product includes instructions directing the user to apply the water insoluble substrates to the face. In a further embodiment, the product includes instructions directing the user to apply one water insoluble substrate to the forehead region of the face and one water insoluble substrate proximate to the mouth region of the face.

Water-Insoluble Substrate

The product of the present invention includes a plurality of water-insoluble substrates, for example, two, three, or four substrates. By “water insoluble” is meant that the substrate, upon immersion in distilled water at 25° C., does not readily dissolve in or readily break apart. The water-insoluble substrate may, however, be disintegrated and/or dissolved slowly, i.e., over a period of several hours up to several days.

A wide variety of materials can be used as the water-insoluble substrate. Examples of suitable substrates include, but are not limited to, non-woven substrates, woven substrates, hydro-entangled substrates, air entangled substrates, natural sponges, synthetic sponges, and polymeric netted meshes.

The water insoluble substrates may be flushable. As used herein, by “flushable” is meant that the substrate will pass through at least 10 feet of waste pipe in two toilet flushes. The material may also be biodegradable.

In one embodiment, the substrates contain a non-woven material. By “non-woven” is meant that the substrate, or a layer of the substrate, is comprised of fibers that are not woven into a fabric but rather are formed into a sheet, mat, or pad layer. The fibers can either be random (i.e., randomly aligned) or they can be carded (i.e., combed to be oriented in primarily one direction. Furthermore, the non-woven substrate can be composed of a combination of layers of random and carded fibers).

Non-woven substrates may be comprised of a variety of natural and/or synthetic materials. By “natural” is meant that the materials are derived from plants, animals, insects, or byproducts of plants, animals, and insects. By “synthetic” is meant that the materials are obtained primarily from various man-made materials or from natural materials, which have been further altered. Non-limiting examples of natural materials useful in the present invention are silk fibers, keratin fibers (such as wool fibers, camel hair fibers) and cellulosic fibers (such as wood pulp fibers, cotton fibers, hemp fibers, jute fibers, and flax fibers).

Examples of synthetic materials include, but are not limited to, those selected from the group containing acetate fibers, acrylic fibers, cellulose ester fibers, cotton fibers, modacrylic fibers, polyamide fibers, polyester fibers, polyolefin fibers, polyvinyl alcohol fibers, rayon fibers, polyurethane foam, and mixtures thereof.

Substrates made from one ore more of the natural and synthetic materials useful in the present invention can be obtained from a wide variety of commercial sources such as Freudenberg & Co. (Durham, N.C. USA), BBA Nonwovens (Nashville, Tenn. USA), PGI Nonwovens (North Charleston, S.C. USA), Buckeye Technologies/Walkisoft (Memphis, Term. USA), and Fort James Corporation (Deerfield, Ill. USA).

Methods of making non-woven substrates are also well known in the art. Such methods include, but are not limited to, air-laying, water-laying, melt-blowing, spin-bonding, or carding processes. The resulting substrate, regardless of its method of production or composition, is then subjected to at least one of several types of bonding operations to anchor the individual fibers together to form a self-sustaining web. The non-woven substrate can be prepared by a variety of processes including hydro-entanglement, thermally bonding, and combinations of these processes. Moreover, the substrates can have a single layer or multiple layers. In addition, a multi-layered substrate can include film layer(s) (e.g., aperture or non-aperture film layers) and other non-fibrous materials.

Strength or firmness of the non-woven material may be a desirable attribute. This can be achieved, for example, by the addition of binding materials, such as wet strength resins, or the material may be made of polymer binder coatings, stable fibres, e.g. based on cotton, wool, linen and the like. Examples of wet strength resins include, but are not limited to, vinyl acetate-ethylene (VAE) and ethylene-vinyl chloride (EVCL) Airflex emulsions (Air Products, Lehigh, Pa.), Flexbond acrylic polymers (Air Products, Lehigh, Pa.), Rhoplex ST-954 acrylic binder (Rohm and Haas, Philadelphia, Pa.), and Ethylene-vinyl acetate (EVA) emulsion (DUR-O-SET® by National Starch Chemicals, Bridgewater, N.J.). The amount of binding material in the substrate may range from about 5% to about 20%, by weight, of the substrate.

Non-woven materials of increased strength can also be obtained by using the so-called spunlace or hydro-entanglement technique. In this technique, the individual fibers are twisted together so that an acceptable strength or firmness is obtained without the need to use binding materials. The advantage of the latter technique is the excellent softness of the non-woven material.

In one embodiment, the non-woven material is made of a superabsorbent polymer. For the purposes of the present invention, the term “superabsorbent polymer” refers to materials which are capable of absorbing and retaining at least about 10 times their weight in body fluids under a 0.5 psi pressure. The superabsorbent polymer particles of the invention may be inorganic or organic crosslinked hydrophilic polymers, such as polyvinyl alcohols, polyethylene oxides, crosslinked starches, guar gum, xanthan gum, and other material known to the art of absorbent article manufacture.

Additives may also be added in order to increase the softness of the substrates. Examples of such additives include, but are not limited to, polyols such as glycerol, propylene glycol and polyethylene glycol, phthalate derivatives, citric esters, surfactants such as polyoxyethylene (20) sorbitan esters, and acetylated monoglycerides.

Sensory attributes may also be incorporated to the insoluble non-woven substrates. Examples of such sensory attributes include, but are not limited to color, texture, pattern, and embossing.

In one embodiment, the product of the present invention includes a plurality of water insoluble substrates of different shapes. In one embodiment, the product includes a first water insoluble substrate and a second water insoluble substrate; wherein the first water insoluble substrate is shaped for application onto the forehead and the second water insoluble substrate is shaped for application proximate to the mouth, such as areas above and/or below the lips, the chin, and/or the cheeks. In one embodiment, the first water insoluble substrate is also applied to the nose region of the face. In one embodiment, the first water-insoluble substrate has a surface area of from about 100 cm² to about 200 cm², such as from about 120 cm² to about 160 cm² and the second water insoluble substrate has a surface area of from about 100 cm² to about 300 cm², such as from about 150 cm² to about 250 cm².

Liquid Carrier

In one embodiment, the product of the present invention incorporates a liquid carrier on the water insoluble substrates. In one embodiment, the liquid carrier is present in at least about 50% by weight of the total weight of the water insoluble substrates. In another embodiment, the liquid carrier is present in less than about 10% by weight of the total weight of the water insoluble substrates. In a further embodiment, the product contains instructions for the user to wet the substrate with water and/or another liquid prior to application. Examples of liquid carriers include, but are not limited to, water, isopropyl alcohol, glycols, hydro-alcohols, glycerin, and esters.

Active Agents

In one embodiment, one or more of the water insoluble substrates include one or more active agents. What is meant by an “active agent” is a compound (e.g., a synthetic compound or a compound isolated from a natural source) that has a cosmetic or therapeutic effect on the skin including, but not limited to, lightening agents, darkening agents such as self-tanning agents, anti-acne agents, shine control agents, anti-microbial agents, anti-inflammatory agents, anti-mycotic agents, anti-parasite agents, external analgesics, sunscreens, photoprotectors, antioxidants, keratolytic agents, detergents/surfactants/self-foaming agents, moisturizers, nutrients, vitamins, energy enhancers, anti-perspiration agents, astringents, deodorants, hair removers, firming agents, anti-callous agents, anti-wrinkle, and skin conditioning agents.

In one embodiment, the agent is selected from, but not limited to, hydroxy acids, benzoyl peroxide, sulfur resorcinol, ascorbic acid and its derivatives, D-panthenol, hydroquinone, octyl methoxycinnimate, titanium dioxide, octyl salicylate, homosalate, avobenzone, polyphenolics, carotenoids, free radical scavengers, spin traps, retinoids such as retinol and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as and coenzyme Q10, lipoic acid, amino acids such a proline and tyrosine, lipo amino acids such as capryloyl glycine and sarcosine, vitamins, lactobionic acid, acetyl-coenzyme A, niacin, riboflavin, thiamin, ribose, electron transporters such as NADH and FADH2, and other botanical extracts, salt, esters, and derivatives thereof. The active agent will typically be present in the composition or product of the invention in an amount of from about 0.001% to about 20% by weight of the composition, e.g., about 0.01% to about 10% such as about 0.1% to about 5%.

Examples of vitamins include, but are not limited to, vitamin A, a vitamin B such as vitamin B3, vitamin B5, and vitamin B12, vitamin C, vitamin K, and vitamin E salts, esters, and derivatives thereof. (e.g., retinyl palmitate, ascorbyl acetate, and tocopherol acetate).

Examples of hydroxy acids include, but are not limited, to glycolic acid, lactic acid, malic acid, salicylic acid, citric acid, and tartaric acid.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbic acid glucoside, magnesium ascorbyl phosphate, and ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isofavonoids and their derivatives (e.g., genistein and diadzein), extracts containing resveratrol and the like. Examples of such natural extracts include grape seed, green tea, pine bark, and propolis. Other examples of antioxidants may be found on pages 1612-13 of the INCI Handbook.

Examples of botanical extracts include, but are not limited to legumes such as Soy, Aloe Vera, Feverfew, Hedychium, Rhubarb, Portulaca, Cedar Tree, Cinnamon, Witch Hazel, Dandelion, Chinese Angelica, Turmeric, Ginger, Burnet, Houttuynia, Coix Seed, and Thyme. What is meant by a “botanical extract” is a blend of two or more compounds isolated from a plant.

In one embodiment, the water insoluble substrate designed for application on the forehead region of the face includes, but is not limited to: oil-control agents such as titanium dioxides, alcohols, botanical extracts, and talc; pore refining agents such as alpha-hydroxy acids, beta-hydroxy acids, and enzymes; anti-acne agents such as benzoyl peroxide, salicylic acid, trichlorcarban, triclosan, azelaic acid, clindamycin, adapalene, erythromycin, sodium sulfacetamide, retinoic acid, and sulfur; oil-absorbing agents such as titanium dioxides and clays; shine control agents such as silicones, alcohols, talc, and clays; dark spot reduction agents such as vitamin C, hydroquinone, botanical extracts, alpha-hydroxy acids, beta-hydroxy acids, and retinoids; and/or wrinkle/fine-line reduction agents such as retinoids, alpha-hydroxy acids, and enzymes.

In another embodiment, the water insoluble substrate that is designed for application around the mouth region of the face includes, but is not limited to: hydration/moisturization agents such a glycerin, silicone, glycols, botanical extracts, and esters; pore-refining agents, anti-acne agents, vasodilators such as niacinamide and horsechesnut extract, vasoconstrictors such as caffeine and botanical extracts; skin-lifting agents such as (e.g., copper containing peptides, dimethyaminoethanol, and polymers); skin-firming polymers, wrinkle/fine-line reduction agents, depigmenting/skin lightening agents such as vitamin C, hydroquinone, botanical extracts, alpha-hydroxy acids, beta-hydroxy acids, retinoids, arbutin, and kojic acid; and depilatory/hair reducing agents such as soy extracts, n-acetyl-cysteine, and isoflavones.

Anti-Acne Agent

In one embodiment, one or more of the water insoluble substrates of the product of the present invention includes an anti-acne agent(s). What is meant by an “anti-acne agent” is a drug product effective in the treatment of acne. Examples of anti-acne agents include, but are not limited to, azelaic acid, clindamycin, adapalene, erythromycin, sodium sulfacetamide, retinoic acid, benzoyl peroxide, sulfur, and salicylic acid. In one embodiment, both the first water insoluble substrate and the second water insoluble substrate include salicylic acid.

In one embodiment, the substrate for application to the forehead and the substrate for application proximate to the mouth include from about 0.01 to about 50 percent, by weight, of the at least one anti-acne agents, e.g., about 0.2 to about 30 percent, by weight, such as about 0.2 to about 15 percent, by weight, of the at least one anti-acne agent.

Alcohol

In one embodiment, the substrate for application on the forehead, of the present invention includes a C₂-C₆ alcohol and/or the substrate for application to area around the mouth does not contain an alcohol. Examples of C₂-C₆ alcohols include, but are not limited to, ethanol and isopropanol.

Other Materials

Various other materials may also be present in the compositions and products useful in the subject invention. These include humectants, emollients, carriers/encapsulation for actives (e.g., liposomes), penetration enhancers, sensory agents (e.g., menthol and methyl lactate), chelating agents (e.g., EDTA) and preservatives (e.g., parabens). In addition, the topical compositions useful herein can contain conventional cosmetic adjuvants, such as dyes, opacifiers (e.g., titanium dioxide and zinc oxide), pigments, and fragrances.

Heating of the Product

In one embodiment, the product may be heated, (e.g., to increase the benefit received by the active agent and to increase the level of comfort achieved by the user). To that end, in one embodiment, the product may include instructions directing the user to place the product in warm water or to expose the product to microwaves.

Packaging of Product

In one embodiment, the product is contained within package, wherein each water insoluble substrate is included within a separate discrete area of such package. Non-limiting examples of material that may be used to manufacture such package include aluminum, polypropylene, polyethylene, and/or polyesters. In one embodiment, the package is substantially air-impermeable.

The following is a description of the manufacture of products of the present invention. Other products can be prepared in an analogous manner by a person of ordinary skill in the art.

### EXAMPLE 1

### T-Zone Mask Composition

The following is an example of a water insoluble substrate for application to the forehead. (“T-shaped water insoluble substrate”). Table I summarizes the composition to be added to the “T” shaped water insoluble substrate.

TABLE I

| | CTFA/INCI Name & | | | % w/w | |
|---|---|---|---|---|---|
| Trade Name | Activity | Function | % w/w | Range | Supplier |
| Glypure | Glycolic Acid-70% | Chemical | 0.65 | 0-10 | Dupont, |
| High | Water-30% | Exfoliator | | | Meguro-ku, |
| Purity | | | | | Tokyo, Japan |
| Sodium | Sodium Citrate | pH Adjuster | 0.95 | 0-1 | Iwata |
| Citrate | | | | | Chemical, |
| | | | | | Iwata City, |
| | | | | | Shizuoka, |
| | | | | | Japan |
| Chelest | Disodium EDTA | Chelating | 0.1 | 0-1 | Chubu Chelest |
| 2BS | | Agent | | | Co., Ltd., |
| | | | | | Yokkaichi |
| | | | | | City, Mie, |
| | | | | | Japan |
| Ajdew N-50 | Sodium PCA-50% | Humectant, | 0.2 | 0-5 | Ajinomoto Co., |
| | Water-50% | Skin | | | Inc., |
| | | Conditioning | | | Chuoh-ku, |
| | | Agent | | | Tokyo, Japan |
| Cedar Tree | Cedrus Atlantica | Oil Control | 0.5 | 0-5 | Alban Muller |
| HG | Bark Extract-.1-.4% | | | | International, |
| | Propylene Glycol40-60% | | | | 93106 |
| | Water-40-60% | | | | Montreuil |
| | | | | | Cedex |
| | | | | | France |
| Witch | Hamamelis | Astringent | 0.5 | 0-5 | Koei Kogyo |
| Hazel | Virginiana Extract-1% | | | | Co., Ltd., |
| Extract | Butylene Glycol-99% | | | | Kanda- |
| | | | | | Awajicho |
| | | | | | Chiyoda-Ku |
| | | | | | Tokyo, 101- |
| | | | | | 0063 |
| | | | | | Japan |
| Houttuynia | Houttuynia Cordata | Skin | 0.2 | 0-5 | Maruzen |
| Extract LA | Extract-0.6% | Conditioning | | | Pharmaceutica |
| | Alcohol-29.82% | Agent | | | Ls Co., Ltd. |
| | Water-69.58% | | | | 14703-10 |
| | | | | | Mukaihigashi |
| | | | | | Onomichi City |
| | | | | | Hiroshima |
| | | | | | 722-0062 Japan |
| Mekkins M | Methylparaben | Preservative | 0.1 | 0-0.5 | Ueno, |
| | | | | | Chuoh-ku, |
| | | | | | Osaka, Japan |
| Alcohol | Alcohol-95% | Cooling | 6 | 0-40 | New Energy and |
| | Water-5% | Agent/Oil | | | Industrial |
| | | Control | | | Technology |
| | | | | | Development |
| | | | | | Org, Minatoku, |
| | | | | | Tokyo, |
| | | | | | Japan |
| Mekkins E | Ethylparaben | Preservative | 0.05 | 0-0.5 | Ueno |
| Salicylic | Salicylic Acid | Exfoliant/ | 0.2 | 0-2 | Duksan, |
| Acid | | Anti-Acne | | | Kyungkido, |
| | | | | | Korea |
| Nikkol | PPG-8-Ceteth-20 | Surfactant | 0.1 | 0-5 | Nikko |
| PBC-44 | | | | | Chemicals |
| | | | | | Chuoku |
| | | | | | Tokyo 103-0002 |
| | | | | | Japan |
| Cooling | Menthoxypropanediol | Cooling agent | 0.02 | 0-1 | Takasago |
| Agents 10 | | | | | Hiratsuka |
| (N) | | | | | City, |
| | | | | | Kanagawa, |
| | | | | | Japan |
| Firmenich | Perfume | Perfume | 0.01 | 0-1 | |
| 475.102/D | | | | | |
| Deionized | Water | Vehicle | 90.42 | Qs to | |
| Water | | | | 100 | |

The Glypure High Purity, sodium citrate, disodium EDTA, Ajdew N-50, cedar tree HG, witch hazel extract, and houttuynia extract LA were combined in a first container with a portion of deionized water at room temperature. In a second container, the methylparaben, ethylparaben, and salicylic acid were mixed with the alcohol at room temperature. PPG-8-Ceteth-20, menthoxypropanediol and perfume were then mixed with the solution of the second container. The solution of the second container was then mixed with the solution of the first container, along with the remainder of deionized water. After achieving a uniformed mixture, the completed mixture was then filtered through a 200-mesh screen.

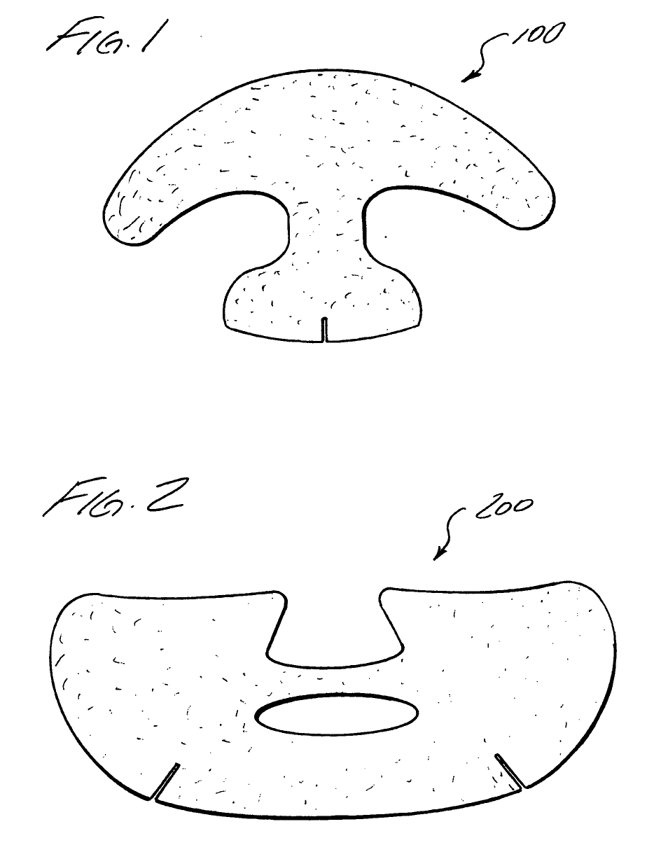

Six grams of the filtered mixture is then added to a “T” shaped water insoluble substrate that was 60 gsm rayon/pulp (55/45) of non-woven fabric supplied by Sansho Shigyo K. K. (Tosa City, Kouchi, Japan). The shape of such substrate 100 is depicted in FIG. 1 and the application of such substrate 100 is depicted in FIG. 3.

### EXAMPLE 2

### U-Zone Mask Composition

The following is an example of a water insoluble substrate for application to the mouth. (“U-shaped water insoluble substrate”) Table II summarizes the composition to be added to the “U” shaped water insoluble substrate.

TABLE II

| | | | | % w/w | |
|---|---|---|---|---|---|
| Trade Name | CTFA/INCI Name | Function | % w/w | Range | Supplier |
| Amisoft MS- | Sodium Myristoyl | Surfactant | .005 | 0-5% | Ajinomoto |
| 11 | Glutamate | | | | |
| Nikkol PBC- | PPG-8-Ceteth-20 | Surfactant | .1 | 0-5% | Nikko |
| 44 | | | | | Chemicals |
| T.I.O. | Triethylhexanoin | Skin | .05 | 0-5% | Nisshinnseiyu, |
| | | conditioning, | | | Chuoh-ku, |
| | | occlusive agent | | | Tokyo, Japan |
| Glypure High | Glycolic Acid- | Chemical | .1 | 0-10% | Dupont |
| Purity | 70% | Exfoliator | | | |
| | Water-30% | | | | |
| Sodium | Sodium Citrate | PH adjuster | .14 | 0-1% | Iwata Chemical |
| Citrate | | | | | |
| Silicone | Cyclomethicone | Emollient | 0.3 | 0-5% | Dow Corning |
| DC246 | | | | | Chiyoda-ku, |
| | | | | | Tokyo, Japan |
| Coix Seed | Coix Lacryma- | Skin | 0.5 | 0-5% | Maruzen |
| Extract BG | Jobi Seed | Conditioning | | | Pharmaceutica |
| | Extract-0.3% | | | | Ls Co., Ltd. |
| | Butylene Glycol- | | | | |
| | 49.85% | | | | |
| | Water-49.85% | | | | |
| Thyme Extact | Thymus Serpillum | Moisturizer, | 0.3 | 0-5% | Maruzen |
| | Extract-0.5% | Astringent | | | Pharmaceutica |
| | Butylene Glycol- | | | | Ls Co., Ltd. |
| | 49.75% | | | | |
| | Water-49.75% | | | | |
| 1,3 Butylene | Butylene Glycol | Humectant | 7.5 | 0-40% | Daicel |
| Glycol | | | | | Chemical, |
| | | | | | Sakai City, |
| | | | | | Osaka, Japan |
| Keltrol | Xanthan Gum | Thickener | .15 | 0-1% | Sansyo, Chuohku, |
| | | | | | Osaka, |
| | | | | | Japan |
| Mekkins M | Methylparaben | Preservative | .1 | 0-0.5% | Ueno |
| Mekkins E | Ethylparaben | Preservative | .05 | 0-0.5% | Ueno |
| Salicylic | Salicylic Acid | Exfoliant/Anti- | .03 | 0-2% | Duksan |
| Acid | | Acne | | | |
| Firmenich | Perfume | Perfume | .01 | 0-1% | |
| 475.102/D | | | | | |
| Chelest 2BS | Disodium EDTA | Chelating Agent | 0.1 | 0-1% | Chubu Chelest |
| | | | | | Co., Ltd. |
| Deionized | Water | Vehicle | 90.5 | Qs to | |
| Water | | | 65 | 100% | |

The xanthan gum was combined with a portion of the butylene glycol in a first container. In a second container, the contents of the first container were then combined with deionized water. To this mixture, sodium citrate, disodium EDTA, coix seed extract BG, thyme extract, and Glypure High Purity were added and mixed at room temperature. Salicylic acid, methylparaben, and ethylparaben were added into another portion of butylene glycol in a third container, which was heated to 60° C.-90° C. Another portion of butylene glycol was added to the third container along with perfume, and the solution was mixed. The contents of the third container were then added to the contents of the second container and then mixed.

In a fourth container, sodium myristoyl glutamate was dissolved in deionized water at 45° C.-55° C. PPG-8-Ceteth-20 was mixed with butylene glycol in a fifth container. The contents of the fourth container were then dissolved into the contents of the fifth container at 30° C.-50° C. Triethylhexanoin and cyclomethicone were then mixed in a sixth container. The contents of the sixth container were then added to the contents of the fifth container. This mixture was then homogenized for 20 minutes at 30° C.-35° C. Deionized water was then slowly added. The mixture was again homogenized for 5 minutes, and the remaining deionized water was added, and the solution was mixed for 10-15 minutes.

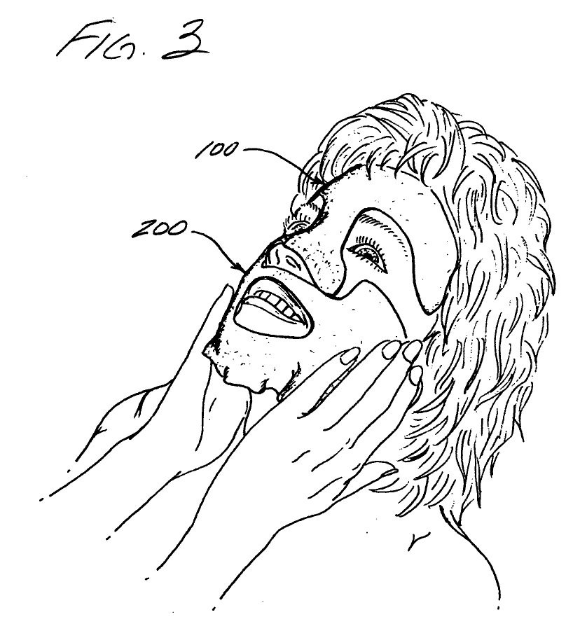

This mixture was then added to the contents of the second container and it was mixed until uniform. The remaining solution was then filtered through a 200 mesh screen. Eleven grams of the composition is then added to a “U” shaped water insoluble substrate that is 60 gsm rayon/pulp (55/45) of non-woven fabric supplied by Sansho Shigyo K. K. The shape of such substrate 200 is depicted in FIG. 2 and the application of such substrate 200 is depicted in FIG. 3.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

1. A product comprising a first water insoluble substrate and a second water insoluble substrate, wherein
(a) said first water insoluble substrate and said second water insoluble substrate are of different shape;
(b) said first water insoluble substrate comprises a first active agent; and
(c) said second water insoluble substrate comprises a second active agent but does not comprise said first active agent.

2. A product of claim 1, wherein the product comprises instructions directing the user to apply the water insoluble substrates to the face.

3. A product of claim 1, wherein said first water insoluble substrate is shaped for application on the forehead and said second water insoluble substrate is shaped for application to an area proximate to the mouth.

4. A product of claim 1, wherein said product comprises instructions directing the user to apply said first water insoluble substrate to the forehead and said second water insoluble substrate to an area proximate to the mouth.

5. A product of claim 1, wherein said first water insoluble substrate and said second water insoluble substrate comprise at least about 50%, by weight of the total weight of the substrate, of a liquid carrier.

6. A product of claim 1, wherein said first water insoluble substrate and said second water insoluble substrate comprise less than about 10% by weight of the total weight of the substrate, of a liquid carrier.

7. A product of claim 4, wherein said first insoluble water substrate and said second water insoluble substrate comprise at least about 50%, by weight of the total weight of the substrate, of a liquid carrier.

8. A product of claim 4, wherein said first water insoluble substrate and said second water insoluble substrate comprise less than about 10% by weight of the total weight of the substrate, of a liquid carrier.

9. A product of claim 1, wherein said second active agent is selected from the group consisting of glycolic acid, salicylic acid, butylenes glycol, coix seed extract, and thymus serpillum extract.

10. A product of claim 1, wherein said first active agent is selected from the group consisting of a C₂-C₆ alcohol, Cedrus Atlanitica Bark Extract, and Hamamelis Virginiana Extract.

11. A product of claim 9, wherein said first active agent is selected from the group consisting of a C₂-C₆ alcohol, Cedrus Atlanitica Bark Extract, and Hamamelis Virginiana Extract.

12. A product of claim 4, wherein said second active agent is selected from the group consisting of glycolic acid, salicylic acid, butylenes glycol, coix seed extract, and thymus serpillum extract.

13. A product of claim 4, wherein said first active agent is selected from the group consisting of a C₂-C₆ alcohol, Cedrus Atlanitica Bark Extract, and Hamamelis Virginiana Extract.

14. A product of claim 12, wherein said first active agent is selected from the group consisting of a C₂-C₆ alcohol, Cedrus Atlanitica Bark Extract, and Hamamelis Virginiana Extract.

15. A product of claim 1 wherein said first water insoluble substrate and said second water insoluble substrate comprises a non-woven material.

16. A product of claim 1, wherein said product further comprises a package comprising a first discrete area and a second discrete area wherein said first water insoluble substrate is contained within said first discrete area and the second water insoluble substrate is contained within the second discrete area.

17. A method of applying a first active agent and a second active agent to the skin, said method comprising the steps of applying a first water-insoluble substrate comprising said first active agent and a second water-insoluble substrate comprising said second active agent to said skin, wherein said second water-insoluble substrate does not comprise said first active agent.

18. A method of claim 17, wherein said first water-insoluble substrate and said second water-insoluble substrate water are applied to the face.

19. A method of claim 18, wherein said first water insoluble substrate is applied to the forehead of the face and said second water insoluble substrate is applied to an area proximate to the mouth.

20. A method of claim 1, wherein said first insoluble water substrate and said second water insoluble substrate comprise at least about 50%, by weight of the total weight of the substrate, of a liquid carrier.
